(19) United States
(12) Patent Application Publication
(10) Pub. No.: US 2024/0225556 A9
(43) Pub. Date: Jul. 11, 2024

(54) FOOT MONITORING METHOD AND SYSTEM

(71) Applicants: UNIVERSITY OF MALTA, Msida (MT); FRIEDMAN, Mark, Ramat Gan (IL)

(72) Inventors: Stephen MIZZI, Naxxar (MT); Owen FALZON, HAZ-ZABBAR (MT)

(21) Appl. No.: 18/279,060

(22) PCT Filed: Feb. 28, 2021
(86) PCT No.: PCT/IB2021/051665

§ 371 Date: Aug. 27, 2023

(51) Int. Cl. A61B 5/00; A61B 5/01; G16H 50/70

(52) U.S. Cl. CPC A61B 5/7267; A61B 5/0008; A61B 5/01; A61B 5/6807; A61B 5/6829; A61B 5/7275; G16H 50/70; A61B 2560/0462; A61B 2562/0219; A61B 2562/0247; A61B 2562/0271; A61B 2562/066

(57) ABSTRACT

An apparatus senses foot conditions of temperature at locations on the plantar and dorsal sides of the foot, and applies computerized processes to provide early detection of foot ulcers and/or vascularization issues, such as artery degradation in the foot.

### TECHNICAL FIELD

The present invention is directed to monitoring systems for feet, in particular for feet of diabetic patients to detect foot conditions common to those with diabetes.

### BACKGROUND

People living with diabetes are subject to increased risks of developing foot complications, such as ulcerations and vascular issues. If not treated, the person may be subject to complex medical treatments and ultimately, leg amputations. Globally, an estimated 422 million adults are living with diabetes mellitus according to the latest 2016 data from the WHO (World Health Organization). Statistics clearly show that in the upcoming decades there shall be a growing need for screening, prevention and management of the disease and its associated complications.

### SUMMARY

The present disclosure (also referred to herein as the disclosed subject matter) is directed to systems and methods for monitoring and detecting foot ulcers and/or vascularization issues, such as artery degradation in the foot, at early stages, so as to be treatable and avoid amputation.

The present disclosure is directed to apparatus which sense foot conditions based on temperature at locations on the plantar and dorsal sides of the foot, and apply computerized processes to provide early detection of foot ulcers and/or vascularization issues, such as artery degradation in the foot.

The present disclosed subject matter provides methods and systems which continuously monitor and analyze abnormal foot temperature patterns, which are indicative of ulcer development, a major concern in the diabetic population as it can lead to severe complications. This is achieved by acquiring dense temperature maps, with over 30 temperature sensors covering both the dorsal and plantar aspect of each foot, providing a drastic improvement with respect to current systems. When combined with the advanced spatial and temporal data analysis techniques being developed and implemented, this leads to significantly improved assessment, prevention and treatment plans for high-risk patients. The system includes a user-friendly mobile application (APP), providing patients with a monitoring tool to alert the patient of possible skin damage during daily activities in real time, for the patient to take precautions as a short term management. It also feeds all information with regard to thermal patterns analysis and activities to the medical consultant in order to be able to provide the patients with a personalized long term management plans.

Embodiments of the present disclosed subject matter include a form factor, such as wearable, typically for the foot, for example, a sock, an insole, wrap, shoe, ankle support, or other foot covering (e.g., full or partial), which includes sensors, in arrays, for measuring foot temperature. The sensor arrays are linked by wired or wireless links to a computer, which analyzes the sensor data to determine early stage ulceration or vascular issues. For example, the sock is typically worn with a shoe, and the in-shoe foot sensor data is obtained.

This document references terms that are used consistently or interchangeably herein. These terms, including variations thereof, are as follows.

A “computer” includes machines, computers and computing or computer systems (for example, physically separate locations or devices), servers, computer and computerized devices, processors, processing systems, computing cores (for example, shared devices), and similar systems, workstations, modules and combinations of the aforementioned. The aforementioned “computer” may be in various types, such as a personal computer (e.g., laptop, desktop, tablet computer), or any type of computing device, including mobile devices that can be readily transported from one location to another location (e.g., a smartphone, personal digital assistant (PDA), mobile telephone or cellular telephone, a watch digitally linked to a network such as the Internet, or other wearable technology such as a digital watch, bracelet or wristband (e.g., a Fitbit™ device) or a Bluetooth headset or other networked headset, or the like.

A “server” is typically a remote computer or remote computer system, or computer program therein, in accordance with the “computer” defined above, that is accessible over a communications medium, such as a communications network or other computer network, including the Internet. A “server” provides services to, or performs functions for, other computer programs (and their users), in the same or other computers. A server may also include a virtual machine or a software based emulation of a computer.

An “application” or “software application”, includes executable software, and optionally, any graphical user interfaces (GUI), through which certain functionalities can be implemented.

A “client” is an application that runs on a computer, workstation or the like and relies on a server to perform some of its operations or functionality.

The terms “n” and “n^{th}” are representative of the last member of a series or sequence of members, for example, servers, databases, computers, elements, with the series being definite or indefinite.

Unless otherwise defined herein, all technical and/or scientific terms used herein have the same meaning as commonly understood by one of ordinary skill in the art to which the disclosure pertains. Although methods and materials similar or equivalent to those described herein may be used in the practice or testing of embodiments of the disclosure, exemplary methods and/or materials are described below. To the extent of any conflict, the patent specification, including definitions, will control. In addition, the materials, methods, and examples are illustrative only and are not intended to be limiting.

### BRIEF DESCRIPTION OF THE DRAWINGS

Some embodiments of the present disclosure (also referred to herein as the disclosed subject matter) are herein described, by way of example only, with reference to the accompanying drawings. With specific reference to the drawings in detail, it is stressed that the particulars shown are by way of example and for purposes of illustrative discussion of embodiments of the disclosure. In this regard, the description taken with the drawings makes apparent to those skilled in the art how embodiments of the disclosed subject matter may be practiced.

Attention is now directed to the drawings, where like reference numerals or characters indicate corresponding or like components. In the drawings:

FIG. 1 is a diagram of an exemplary system in accordance with embodiments of the present disclosed subject matter;

FIG. 2 is a diagram of sensor arrays on the foot receiving device of the system of FIG. 1;

FIG. 3 is a block diagram of the system of FIG. 1;

FIGS. 4-11 are flow diagrams of an exemplary process performed by the system of FIG. 1;

FIG. 12 is a flow diagram of an exemplary process performed by the system of FIG. 1 with data received from the processes of FIGS. 4-11 respectively; and,

FIG. 13 is a Table listing the status and condition applicable for each of the Diagrams of FIGS. 4-11.

### DETAILED DESCRIPTION OF THE DRAWINGS

Before explaining at least one embodiment of the disclosed subject matter in detail, it is to be understood that the disclosure is not necessarily limited in its application to the details of construction and the arrangement of the components and/or methods set forth in the following description and/or illustrated in the drawings. The disclosed subject matter is capable of other embodiments or of being practiced or carried out in various ways.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more non-transitory computer readable (storage) medium(s) having computer readable program code embodied thereon.

Throughout this document, numerous textual and graphical references are made to trademarks. These trademarks are the property of their respective owners, and are referenced only for explanation purposes herein.

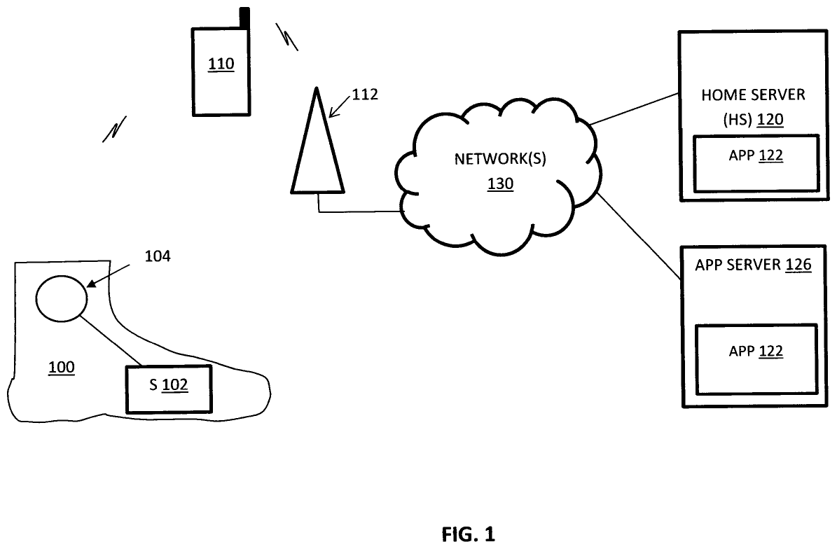

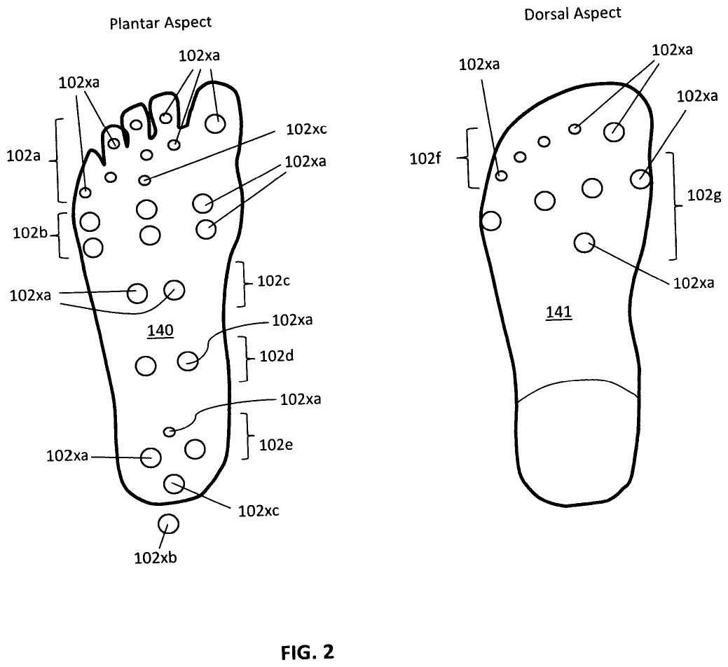

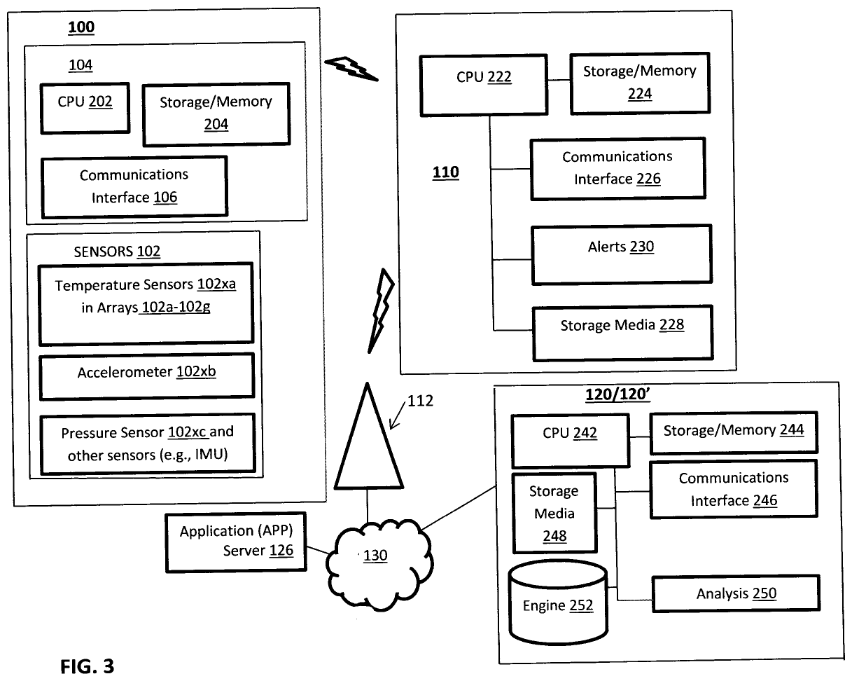

FIG. 1 shows a system in accordance with the disclosed subject matter. The system includes a foot receiving device or form factor 100, such as wearable, typically for the foot, for example, a sock, an insole, wrap, shoe, which includes sensors (S) 102, which can be arranged in arrays 102a-102g, linked to a data processing and communication unit 104. The sensors 102 include one or more of temperature sensors 102xa, accelerometers 102xb or other sensors 102xc, such as pressure sensors, internal measurement units (IMUs) and the like, as shown in FIGS. 2 and 3.

The foot receiving device 100 links to a mobile computer 110, such as a smart phone, tablet, iPad® or the like, by Bluetooth® or other local communication system. The mobile computer 110 (via a cellular tower 112) links to a home or main server (also known as a home or main computer) 120 over a network(s) 130. The home server 120 may also store an application (APP) 122, detailed below, accessible by the smart phone 110, for download to the smartphone 110. Alternately, the Application (APP) 122 is available on an Application Server 126, linked to the network(s) 130.

The application (APP) 122, once installed and running (executing) on the mobile computer 120, may include a graphical user interface (GUI) with a display. The Application Server 126, for example, may also be operative, to process sensor 102 data (e.g., data received from the various sensors 102xa, 102xb, 102xc of the arrays 102a-102g or individually (if the sensor is not part of an array)), with the processed outcome being sent to the mobile computer associated with the particular user 110, and/or the home server 120, e.g., the system 120′ of the home server 120.

Additionally, for example, the mobile computer 110 of the user may also be operative, to process sensor 102 data (e.g., data received from the various sensors 102xa, 102xb, 102xc of the arrays 102a-102g or individually (if the sensor is not part of an array)), which the mobile computer 110 receives, with the processed outcome being additionally sent to the home server 120 and/or the Application Server 126. In this case, where the mobile computer 110 is the sensor data receiver and/or collector and data processor, further data processing, such as analysis techniques that may be more computationally-intensive or memory-intensive (potentially not supported by the mobile computer), or analysis techniques that require models not available on the user's mobile computer, may be performed on the home server 120 and/or the application server 126.

The network 130 is, for example, a communications network, such as a Local Area Network (LAN), or a Wide Area Network (WAN), including public networks such as the Internet. As shown in FIG. 1, the network 130, may be a single network, such as the Internet, but is typically a combination of networks and/or multiple networks including, for example, cellular or Bluetooth® or other networks. “Linked” as used herein includes both wired or wireless links, either direct or indirect, for placing the computers, including, servers, and their components, computer components and the like, into electronic and/or data communications with each other.

FIG. 2 is a schematic view of the foot receiving device 100, such as a sock, with the various sensors 102xa (temperature), 102xb (acceleration), 102xc (pressure) (collectively indicated as element 102 in FIG. 1) positioned therein in sensor arrays 102a-102g, with the accelerometer 102xb not part of an array, for example. In the arrays 102a-102g, for example, all of the individual sensors are temperature sensors 102xa, except where other sensors, e.g., accelerometer 102xb, pressure sensor 102xc, or other sensor, are specifically indicated. The sensors 102 are, for example, in groups or arrays 102a-102g, with arrays 102a-102e on the plantar area 140 of the foot, and arrays 102f and 102g on the dorsal area 141 of the foot. The arrays are such that sensors 102 are positioned corresponding to the toes in an array 102a, ball of the foot in an array 102b, front arch in an array 102c, rear arch in an array 102d, heel in an array 102e, and, an accelerometer 102xb at the Achilles. On the dorsal area 141 of the foot, sensors arrays are positioned corresponding to the toes with array 102f, and frontal foot with array 102g. Other sensors, such as the accelerometer 102xb and pressure sensors 102xc, may be part of an array or may be separate from an array. Arrays may comprise one or more sensors 102xa, 102xb, 102xc. These sensors 102xa, 102xb, 102xc, typically in arrays 102a-102g, coupled with individual sensors, and/or any individual sensors, for example, are linked, by wired and/or wireless links to the data processing and communication unit 104. Additionally, for example, the aforementioned temperature sensors and/or accelerometers, pressure sensors and other sensors, may be part of or placed in the data processing and communication unit 104.

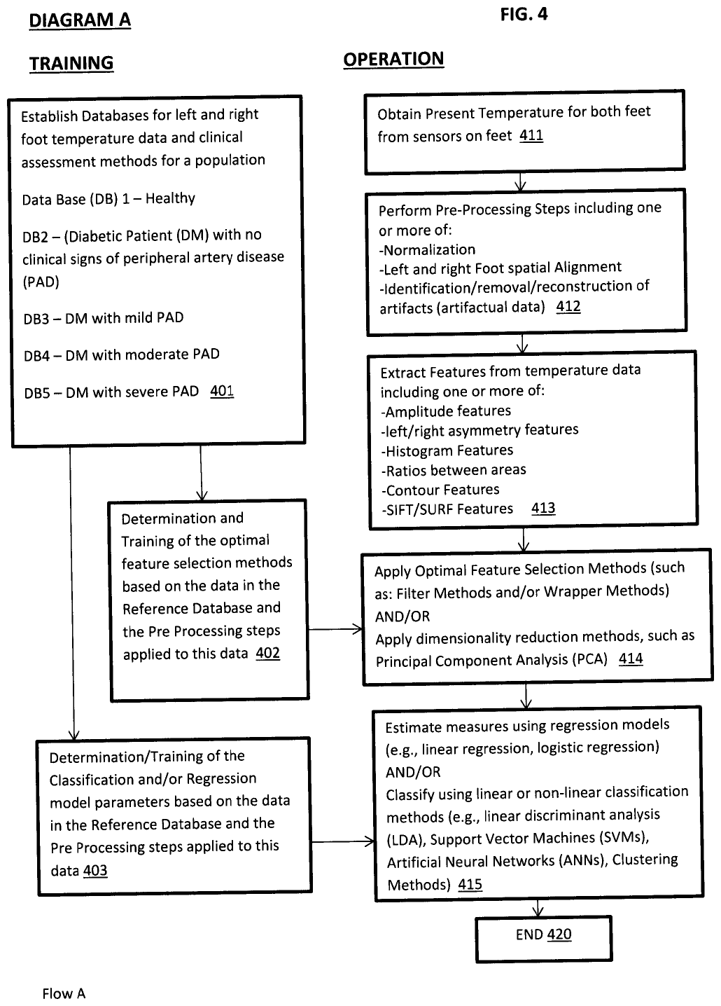

FIG. 3 is a block diagram of the architectures of elements of the overall system of the disclosed subject matter, including, for example, the foot receiving device 100 (and its associated electronics), the mobile computer 110 and the home server 120. The home server 120 includes a system 120′, as shown in the home server 120, which is exemplary only. This is because one or more of the components, modules, engines, and the like of the system 120′ may be external to the home server 120 including in the cloud. As used herein, a “module”, for example, includes a component for storing instructions (e.g., machine readable instructions) for performing one or more processes, and including or associated with processors, e.g., the CPU 202, for executing the instructions. As used herein, an “engine” performs one or more functions, and may include software programs for performing the processes detailed herein and shown in FIGS. 4-12. Other components are also permissible in the foot receiving device 100, the mobile computer 110 and the home server 120. The foot receiving device 100, the mobile computer 110 and the home server 120, and all components in the foot receiving device 100, the mobile computer 110, and the home server 120, are linked to and in communication with each other, either directly or indirectly.

The foot receiving device 100 includes sensors 102, which include sensor arrays 102a-102g, typically formed of temperature sensors 102xa, accelerometers 102xb, individually, as a part of an array, or as an individual array of accelerometers, and pressure 102xc, or other sensors, individually, as a part of an array, or as an individual array of pressure or other sensors. There is also a data processing and communications unit 104, formed of a processor based central processing unit (CPU) 202. The CPU 202 is linked to storage/memory 204 and a communications interface 206.

The Central Processing Unit (CPU) 202 is formed of one or more processors, including microprocessors, for performing sensor 102 and communications interface 106 functions and operations detailed herein. The processors are, for example, conventional processors, and hardware processors, such as those used in servers, computers, and other computerized devices. For example, the processors may include x86 Processors from AMD (Advanced Micro Devices) and Intel, Xenon® and Pentium® processors from Intel, as well as any combinations thereof.

The storage/memory 204 stores machine executable instructions for execution by the CPU 202, to perform the processes of the foot receiving device 100, such as temperature measurements, accelerometer measurements, pressure measurements, and other measurements. The storage/memory 204 also includes storage media for temporary storage of data. The storage/memory 204 also includes machine executable instructions associated with the operation of the sensors 102 and the communications interface 106. The communications interface 206 is, for example, configured for Bluetooth® communications with the mobile computer 110, e.g., smart phone.

The mobile computer or smart phone 110 includes a CPU 222 linked to storage/memory 224, similar to those elements 202 and 204 detailed above. The CPU 222 controls a communications interface 226, which is linked thereto. The CPU 222 also links to storage media 228, and an alert module 230, which issues alerts based on the executing software of the application detecting a condition for which the user needs to be made aware of.

The home server 120 includes a CPU 242 linked to storage/memory 244, similar to those elements 202, 222 and 204, 224 detailed above. The CPU 242 controls a communications interface 246, which is linked thereto. The CPU 242 also links to storage media 248, an analysis module 250, and an engine 252.

The analysis module 250, for example, analyzes the received data, and uses algorithms to detect conditions such as early ulceration and arterial problems. The Application Server 126 as well as the mobile computer 110, for example, may also include modules and/or be programmed similar to that of the analysis module 250 to analyze the received data, and use algorithms to detect conditions such as early ulceration and arterial problems.

The engine 252, performs functions including, for example, one or more of model selection, model training, classification and analysis of values, parameters and/or data to determine conditions, for example, diabetic conditions, including the extent (level) of the condition, and the progression of the condition, is performed, for example, fully or partially by the engine 252. The engine 252, for example, also performs processes associated with a rules and logic based approach, or artificial intelligence (AI), to derive output from a knowledge base. The engine 252, for example, operates with Support Vector Machines (SVMs), Artificial Neural Networks (ANNs).

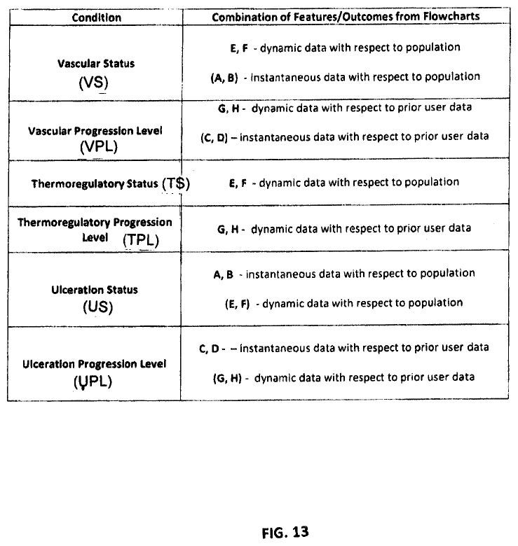

Attention is now directed to FIGS. 4-11, which show flow diagrams, DIAGRAMS A-H, detailing computer-implemented processes in accordance with embodiments of the disclosed subject matter. Reference is also made to elements shown in FIGS. 1-3. The process and sub-processes of FIGS. 4-11 are computerized processes performed by the foot receiving device 100, the mobile computer 110, the home server 120, and/or the application server 126. The aforementioned processes and sub-processes can be, for example, performed automatically, and, for example, in real time. The aforementioned processes of DIAGRAMS A-H are used to determine various status and progression levels of diabetic conditions, with the specific diagrams for each status and progression level listed in FIG. 13.

The system 120′ of the home server 120 obtains readings, for example, of data and/or values, such as temperature, acceleration, pressure, and/or other sensor values, for example, as raw data, from the respective sensors of the respective arrays, for example, arrays 102a-102g, and if part of an array or otherwise on the device, accelerometers 102xb and/or pressure 102xc or other sensors. The raw data is converted or otherwise transformed, into a single value, but may be plural values, which may be, for example, normalized values. The normalized values are obtained, for example, in accordance with the “Normalization” detailed as follows. The normalization is performed, for example, as part of the data processing by the CPU 242.

Normalization

The following are example normalization procedures, that can be adopted to normalize the temperature data recorded from each of the sensor arrays, for example, arrays 102a-102g of FIG. 2:
1. In the case of internal-foot temperature normalization, the temperatures in the temperature array for a given foot can be normalized with respect to the maximum and minimum temperature values in that array.
   For example, if the temperature from one of the sensors (of the array) is denoted by x, the normalized temperature for that sensor, denoted by xₙₒᵣₘ, can be obtained by normalizing x with respect to the maximum temperature, xₘₐₓ, and the minimum temperature, xₘᵢₙ, in the array, such that:

$$\\ {x}_{norm} = \frac{x - {x}_{min}}{{x}_{max} - {x}_{min}}$$

The same normalization concept may, for example, be extended to consider the combined temperature arrays from both feet.
2. In the case of temperature normalization over time, the temperature for a given sensor, is normalized with respect to the maximum and minimum temperature values for that sensor over a considered period of time.
   For example, if the temperature for a given sensor at a particular time point, t, is denoted by xₜ, the normalized temperature for that sensor over time, denoted by xₜ_{ₙₒᵣₘ}, can be obtained by normalizing xₜ with respect to the maximum temperature, xₜ_{ₘₐₓ}, and the minimum temperature, xₜ_{ₘᵢₙ}, for that same sensor over the considered time interval, such that:

$$\\ {x}_{{t}_{norm}} = \frac{{x}_{t} - {x}_{{t}_{min}}}{{x}_{{t}_{max}} - {x}_{{t}_{min}}}$$

The same normalization concept may, for example, be extended to consider the combined temperature from multiple sensors or both feet.

The values, e.g., normalized values, are analyzed including compared to known values and/or ranges taken from a population and/or other source of data, for example, as a data set for training models and the like, to establish a condition, and the extent of the condition, and trained into the system 120′ by various training methods. The training methods, for example, are such that a mapping from the input data to the output data needs to be determined. In the case of a model consisting of an artificial neural network, an example of a widely adopted approach consists of the stochastic gradient descent algorithm, where model parameters are updated during a number of iterations with the use of the backpropagation algorithm. Additionally, for example, the training is such that the training input and training output data is used to update model parameters over a number of iterations.

The condition, for example, may be a medical condition, such as a diabetic condition, e.g., vascular status (VS), thermoregulatory status (TS) and ulceration status (US), and the severity or extent of the condition, such as, Severely Compromised, Moderately Compromised, Marginally Compromised, or Uncompromised. When values are obtained, for example, over a time period, such as taken at intervals, both regular and/or random, or combinations thereof, the condition and the progression of the condition, based on a range of known values, for example a data set, taken from a population (and trained into the system 120′—for example, the data set being used as training data for a selected model), can be analyzed and, in many cases, determined. The population from which the data set is obtained, is, for example, both healthy and diabetic individuals, in order to obtain a broad range of values, representative of various diabetic conditions and condition progressions, and the status or levels of the conditions and condition progressions. The conditions include, for example, medical conditions, such as the diabetic medical conditions of, for example, vascular status (VS), thermoregulatory status (TS) and ulceration status (US), resulting from the diabetic conditions of vascular conditions, thermoregulatory conditions, and ulceration, respectively, and the severity of the aforementioned diabetic medical condition includes at least one of: uncompromised, marginally compromised, moderately compromised, and severely compromised. The progression levels of each of these diabetic medical conditions, are for example, a vascular progression level (VPL), a Thermoregulatory Progression Level (TPL), and an Ulceration Progression Level (UPL), resulting from the diabetic conditions of vascular conditions, thermoregulatory conditions, and ulceration, respectively, with the actual status (progression or progression levels) of the indicated progression levels, including, for example, Severe Deterioration, Moderate Deterioration, No Change, Moderate Improvement, and Strong Improvement.

In the flow diagrams of FIGS. 4-11, model selection, training of models or analysis of conditions may be performed with regression models and/or classification models. The regression models are, for example, linear regression and logistic regression. The classification models, for example, use linear or non-linear classification methods, such as linear discriminant analysis (LDA), Support Vector Machines (SVMs), Artificial Neural Networks (ANNs), Clustering Methods). The aforementioned model selection, model training, classification and analysis of values, parameters and/or data to determine conditions, for example, diabetic conditions, including the extent (level) of the condition, and the progression of the condition, is performed, for example, fully or partially by the engine 252.

FIG. 4 is a Flow Diagram, DIAGRAM A, for a process for evaluating left and right foot temperatures of users for instantaneous data with respect to a population, to determine vascular status and ulceration status. DIAGRAM A includes a Training Phase, blocks 401-403 and an Operation Phase, blocks 411-415, terminating at an END, at block 420.

The Training Phase is made, for example, on the temperature sensing array data acquired (as known data in a data set) from a population of healthy and diabetic participants. For this phase, both the sensor data and the corresponding patient ankle brachial pressure index (ABPI), toe brachial pressure index (TBPI), or other indices that provide information on the vascular status of the lower limbs of the user (patient). The data from the temperature sensing arrays 102a-102g and the clinical measures (e.g., ABPI, TBPI) are gathered from individuals, e.g., healthy and diabetic at various stages, and stored in databases and other storage media, referred to in the DIAGRAMs herein.

In the Training Phase at the left side of the page (DIAGRAM A), databases are established for various temperature ranges corresponding to left and right foot temperature data, and clinical assessment methods in a population, at block 401. For example, at block 401 five databases (DB), indicated as DB1 through DB5 are established for foot data temperature ranges, corresponding to Peripheral Artery Disease (PAD) in the feet, as follows:
DB1—Healthy
DB2—Diabetic Patient (DM) with no clinical signs of PAD
DB3—DM with Mild PAD
DB4—DM with Moderate PAD
DB5—DM with Severe PAD

From block 401, training at block 402, includes, for example, determination and training of the optimal feature selection methods, including, for example models based on the data in the reference database and the preprocessing steps are applied to this data.

Also from block 401, the process moves to block 403, where a classification and/or regression model is determined and/or trained with parameters based on data (data set from a population) in a reference database, for example DB1 to DB5, which includes, for example, temperature data previously acquired from the system's sensors over time, during ambulation, at rest, or during other activities, from healthy individuals and individuals living with diabetes with different types/levels of severity of complications, such as vascular issues, ulceration issues and thermoregulatory issues. Additionally, preprocessing steps, are a normalization with respect to a temperature scale (i.e., rescaling of the input temperatures from the system's sensors to a constrained range of values), temporal normalization (i.e., rescaling the temperature data from the system's sensors to a set constrained range of values over an interval of time), spatial realignment of left and right foot temperature data, identification/removal/reconstruction of artifacts, or artefactual data, and subspace decomposition methods.

Turning to the Operational side, at the right side of the page, at block 411, the present temperature for both feet is obtained. Moving to block 412, one of more preprocessing steps is performed on the obtained temperatures to convert the temperatures into data used by the system.

These preprocessing steps include one or more of: normalization with respect to a temperature scale (i.e., rescaling of the input temperatures from the system's sensors to a constrained range of values), temporal normalization (i.e., rescaling the temperature data from the system's sensors to a set constrained range of values over an interval of time), spatial realignment of left and right foot temperature data, identification/removal/reconstruction of artifacts (artefactual data), and subspace decomposition methods.

At block 413, features extracted from the temperature data include one or more of: amplitude features, left/right foot asymmetry features, histogram features, Scale Invariant Feature Transformation (SIFT)/Speeded Up Robust Features (SURF) features, for example, as detailed in “Speeded Up Robust Features” in Wikipedia at https://en.wikipedia.org/wiki/Speeded_up_robust_features, and contour features.

Next, using the determination and training of the optimal feature selection methods of block 402, and from block 413, the process moves to block 414. At block 414, optimal features selection methods, for example, which may be filter and/or wrapper methods may be used, and/or dimensionality reduction methods, such as principal component analysis (PCA), may be applied.

Next, using the determination/training of the classification/regression model of block 403, and from block 414, the process moves to block 415. At block 415, measures representative of the foot condition and foot condition progression levels are estimated using, for example, regression methods, e.g., linear regression or logistic regression; and/or features related to the foot condition/progression level are classified using linear or non-linear classification methods. These methods may include, for example, linear discriminant analysis (LDA), support vector machines (SVMs), and, artificial neural networks (ANN), including convolutional neural networks (CNNs), and clustering methods.

A linear regression model in its simplest form can be considered as a simple linear regression problem, where an output value, y, representative of the vascular status, is estimated from:

y=β₀+β₁x₁+β₂x₂ . . . +βₙxₙ

in which, xᵢ represents the input temperature data from sensor i. The coefficients βᵢ, provide the mapping between the inputs xᵢ and the predicted output y. The coefficients can be, for example, determined by minimizing the sum of square differences between the estimated output values yₖ^{p}, and the actual values yₖ

$$\\ {\sum }_{k = 1}^{N} {( {y}_{k} - {y}_{k}^{{}^{} p} )}^{{}^{} 2}$$

In the context of vascular status, the values for yₖ used for the determination of the model coefficients can be obtained from clinical measures such as the ankle-brachial pressure index (ABPI). The ABPI values (or other quantitative clinical measures) can be mapped to a scale reflecting the vascular state in relation to the adopted Foot Health Profile.

The clinical measures can be mapped to a range of values with a lower and upper limit of 0 and 1 respectively, where a value of 0 represents an uncompromised foot condition, and a value of 1 would corresponding to a severely compromised case.

Alternatively, a convolutional neural network (CNN) can also be considered to map the temperature data from the foot sensing array to an output value representing vascular status. In the case of instantaneous data, the input to the network can consist of an array of M input temperature values corresponding to the sensors from one foot. Similarly, a model with an input array consisting of 2M elements, which takes into consideration temperature data from both feet (thereby taking into consideration potential relationships between the left and right foot) can also be considered.

The above can also be extended to take into account dynamic temperature data, through a concatenation of multiple arrays obtained T time intervals apart. Models at different temporal scales can be considered to take into account the short term (e.g. same day), medium term (e.g. over day/weeks), and long term changes (over months).

An example of a convolutional neural network that can be used for vascular status estimation can consist of a combination of convolutional layers, pooling layers, and fully connected layers. For example, the network architecture can comprise two convolutional blocks in sequence, each consisting of a convolution layer and a max pooling layer, which are then followed by a fully connected layer to obtain a value for the vascular status estimate.

The convolutional layers extract features from the sensor data such as temperature gradient orientations. The pooling layers are used to reduce the spatial size of the output from the convolution layers. The output of the convolutional and pooling layer then serves as input to one (or more) fully connected layer of neurons.

Rectified linear activation units (RELU) functions given by:

ƒ(x)=max(0,x)

or leaky RELU functions given by:

$$\\ f ( x ) = \{ \begin{matrix}x & if x > 0 \\ ax & otherwise\end{matrix}$$

can be employed as activation units for the network.

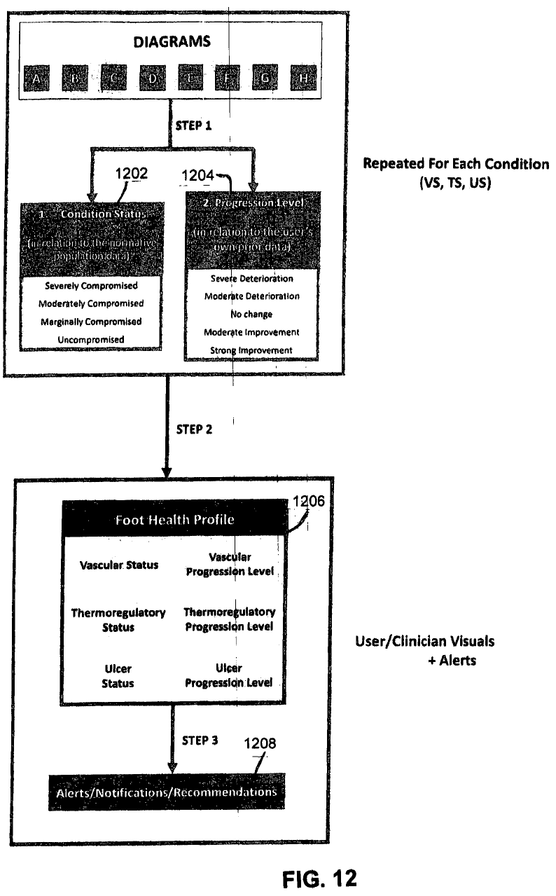

From block 415, the process moves to block 420, where it ends. The resultant data is now ready for analysis by the process of FIG. 12.

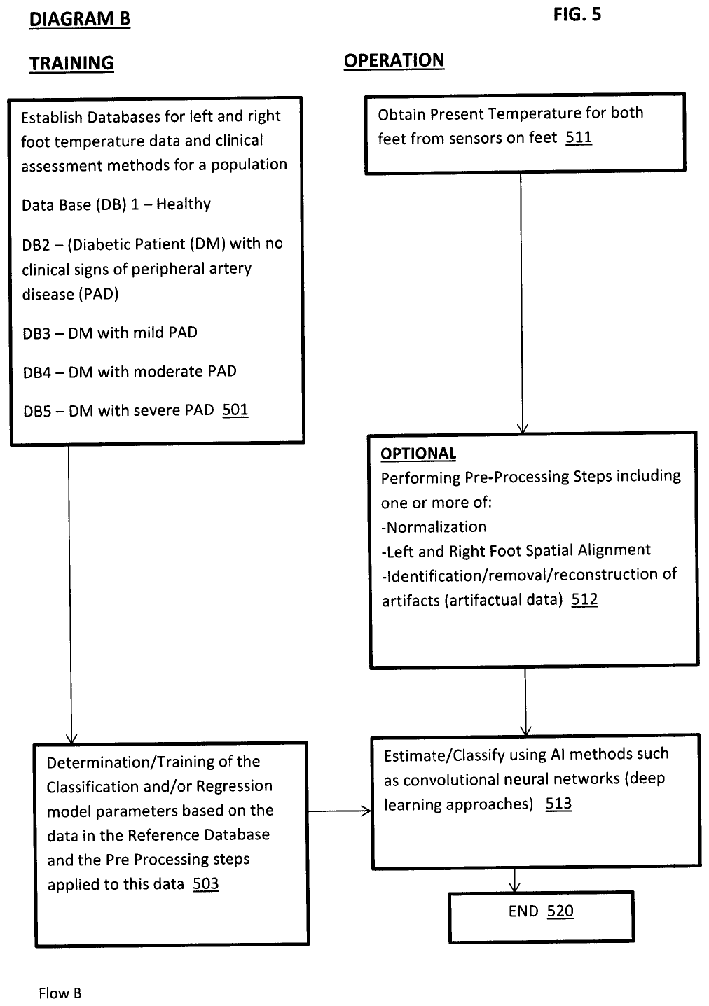

Attention is now directed to FIG. 5, and DIAGRAM B, a process for determining vascular status. The diagram includes a Training Phase, blocks 501-503 and an Operation Phase, blocks 511-513, terminating at an END, at block 520.

The training phase is such that blocks 501 and 503 are identical to blocks 401 and 403, respectively, and are in accordance with the descriptions of the blocks 401 and 403, for FIG. 4.

In the operational phase, at block 511, the present temperature for both feet is obtained from the sensors 102 of the device.

Next, using the determination and training of the optimal feature selection methods of block 502, and from block 511, the process moves to block 512. The process of block 512 is an optional process, and need not be performed when preprocessing is not needed. At block 512, when preprocessing is needed, the preprocessing steps to be performed include, for example, one or more of normalization, left and right foot spatial alignment, and, identification/removal/reconstruction of artifacts (artefactual data).

Next, using the determination/training of the classification/regression model of block 503, and from block 512 (if preprocessing steps are performed), or block 511 (if preprocessing steps were not necessary), for example, when raw data is suitable for use in the models), the process moves to block 513. At block 513, the temperature data following any pre-processing is input into a regression and/or classification model, and from this, the status or progression level of the considered level can be estimated and/or classified, for example, by using artificial intelligence (AI) methods, such as convolutional neural networks using deep learning approaches.

From block 513, the process moves to block 520, where it ends. The resultant data is now ready for analysis by the process of FIG. 12.

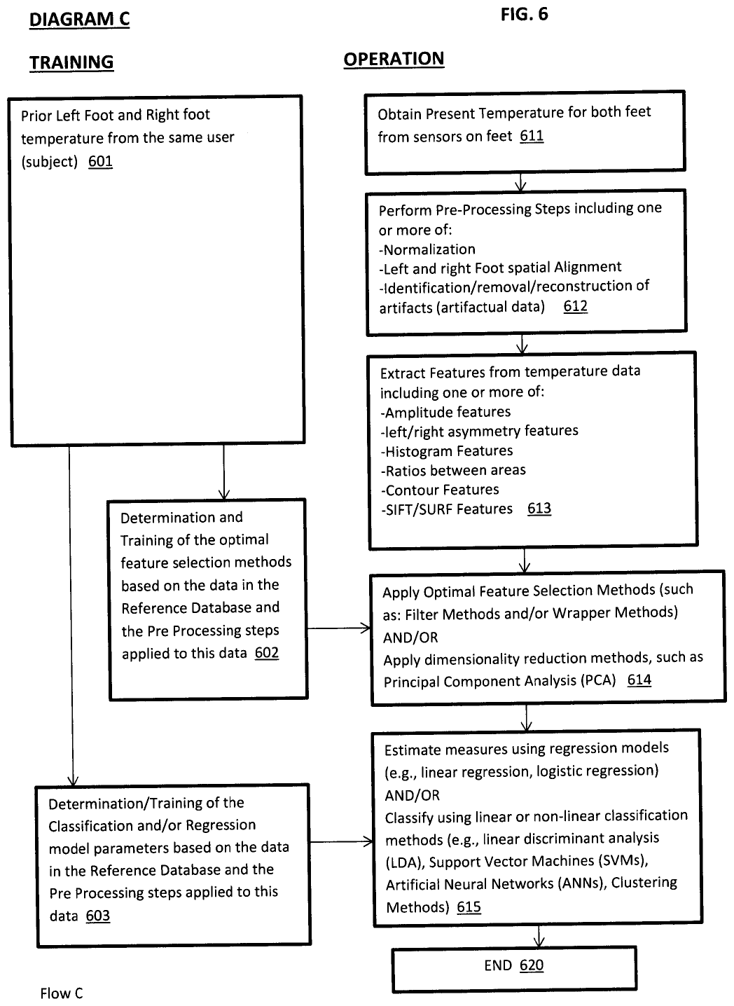

Attention is now directed to FIG. 6, and DIAGRAM C, a process for determining instantaneous data with respect to prior user data for Vascular Progression Levels and Ulceration Progression Levels. The diagram includes a Training Phase, blocks 601-603 and an Operation Phase, blocks 611-615, terminating at an END, at block 620.

In the Training Phase, at the left side of the page, the process begins at block 601, where prior left and right foot temperatures for the same user (subject or patient) are obtained. The process of the training phase moves to block 602, which is similar to block 402 as described for FIG. 4 above. The process then moves to block 603, which is similar to block 403 as described for FIG. 4 above.

In the operational phase, on the right side of the page, the process includes blocks 611, 612, 613, 614 and 615. These processes are similar to corresponding blocks 411, 412, 413, 414 and 415 of FIG. 4, respectively, and are in accordance with these blocks as described for FIG. 4 above.

From block 615, the process moves to block 620, where it ends. The resultant data is now ready for analysis by the process of FIG. 12.

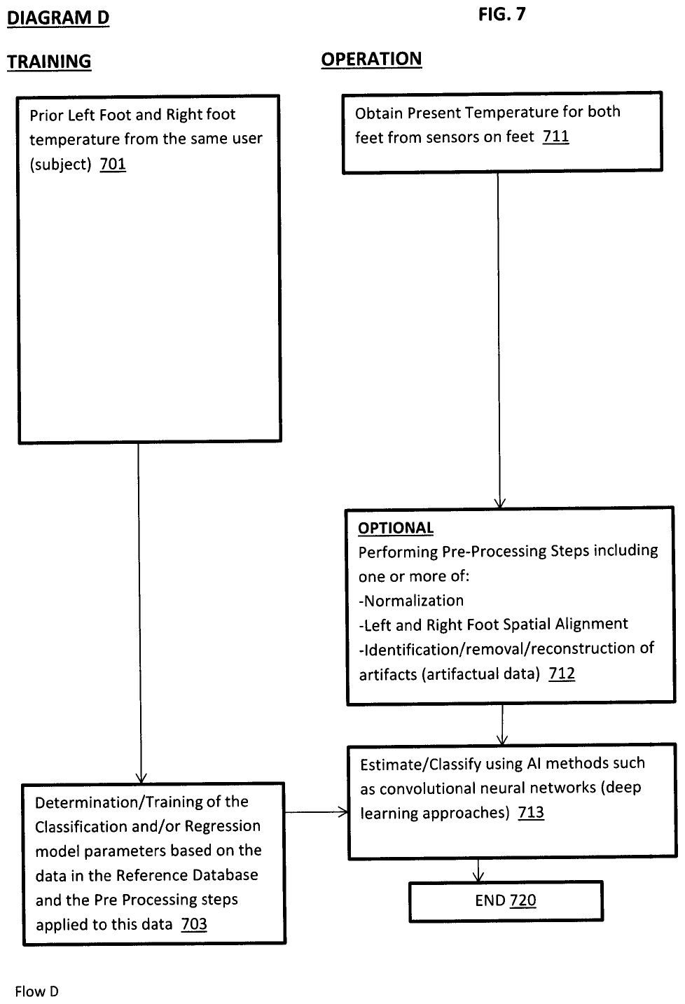

Attention is now directed to FIG. 7, and DIAGRAM D, a process for determining instantaneous data with respect to prior user data for Vascular Progression Levels and Ulceration Progression Levels. The diagram includes a Training Phase at blocks 701 and 703, and an Operation Phase, blocks 711-713, with block 712 being optional should preprocessing be necessary, terminating at an END, at block 720.

In the Training Phase at the left side of the page, the process begins at block 701, where similar to block 601, prior left and right foot temperatures for the same user (subject or patient) are obtained. The process then moves to block 703, which is similar to blocks 403 and 603, as described for FIG. 4 and FIG. 6 above.

In the operational phase, on the right side of the page, the process includes blocks 711, 712 and 713. These processes are similar to corresponding blocks 511, 512 and 513 of FIG. 5, respectively, and are in accordance with these blocks as described for FIG. 5 above.

From block 713, the process moves to block 720, where it ends. The resultant data is now ready for analysis by the process of FIG. 12.

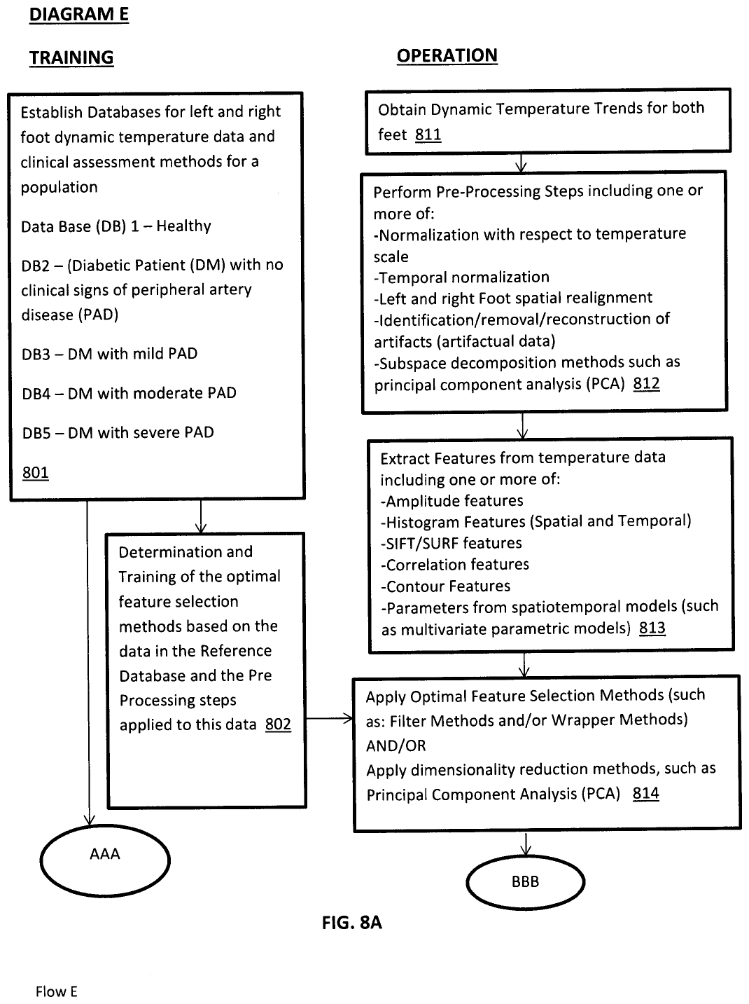

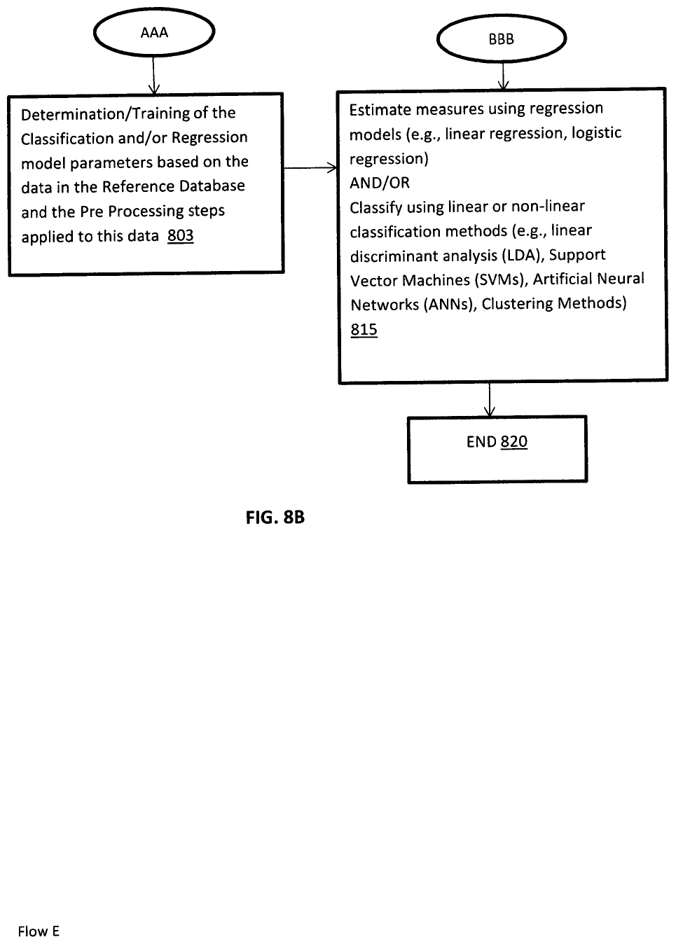

Attention is now directed to FIGS. 8A and 8B, collectively referred to as FIG. 8, and DIAGRAM E, a process for determining vascular status and thermoregulatory status using dynamic data. Dynamic data is, for example, -temperature data acquired from the system's sensors over time, during ambulation, at rest, or during other activities. The diagram includes a Training Phase at blocks 801-803, and an Operation Phase at blocks 811-815, terminating at an END, at block 820.

The training phase is such that at block 801, databases are established for left and right foot dynamic temperature data and clinical assessment methods for a population. The databases (DB) are as follows:
DB1—Healthy
DB2—Diabetic Patient (DM) with no clinical signs of PAD
DB3—DM with Mild PAD
DB4—DM with Moderate PAD
DB5—DM with Severe PAD

Blocks 802 and 803 are identical to blocks 402/602 and 403/603, respectively, and are in accordance with the descriptions of the blocks 402/602 and 403′603, for FIG. 4 and FIG. 6, respectively.

In the operational phase, at block 811, dynamic temperature trends for both feet, left and right, are obtained, for example, from data acquired from system users, typically other diabetic patients at various stages of the disease. Moving to block 812, one of more preprocessing steps is performed on the obtained temperatures to convert the temperatures into data used by the system. These preprocessing steps include one or more of: normalization with respect to a temperature scale (i.e., rescaling of the input temperatures from the system's sensors to a constrained range of values), temporal normalization (i.e., rescaling the temperature data from the system's sensors to a set constrained range of values over an interval of time), spatial realignment of left and right foot temperature data, identification/removal/reconstruction of artifacts (artefactual data), and subspace decomposition methods, such as principal component analysis (PCA).

At block 813, features extracted from the temperature data may, for example, include one or more of: amplitude features, histogram features (spatial and temporal), SIFT/SURF features, correlation features, contour features, and parameters, such as from spatiotemporal models (e.g., multivariate parametric models).

Next, using the determination and training of the optimal feature selection methods of block 802, and from block 813, the process moves to block 814. At block 814, optimal features selection methods, for example, which may be filter and/or wrapper methods may be used, and dimensionality reduction methods, such as principal component analysis (PCA), are, for example, applied.

Next, using the determination/training of the classification/regression model of block 803, and from block 814, the process moves to block 815. At block 815, for example, vascular status may be estimated using regression models and/or methods, e.g., linear regression or logistic regression; and/or classified (e.g., classification models) using linear or non-linear classification methods. These methods include, for example, linear discriminant analysis (LDA); support vector machines (SVMs), artificial neural networks (ANN), and clustering methods.

From block 815, the process moves to block 820, where it ends. The resultant data is now ready for analysis by the process of FIG. 12.

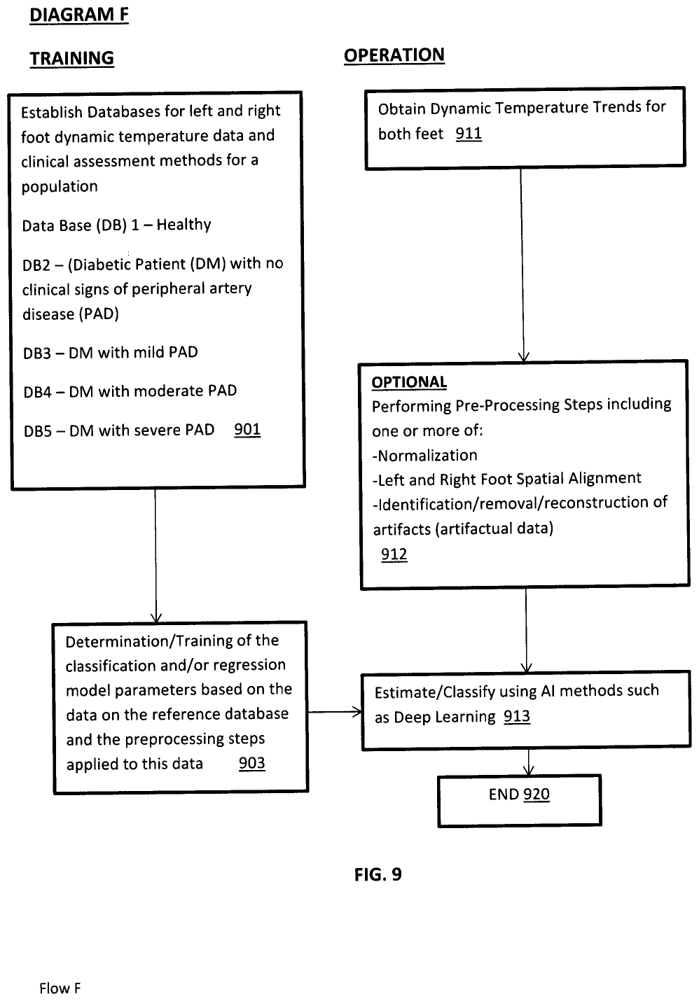

Attention is now directed to FIG. 9, and DIAGRAM F, a process for determining vascular status (VS), thermoregulatory status (TS), and ulceration status (US) based on dynamic data with respect to a population. The diagram includes a Training Phase at blocks 901 and 903, and an Operation Phase at blocks 911-913, with block 912 being optional if preprocessing is needed, and terminating at an END, at block 920.

The training phase, at block 901, includes obtaining dynamic temperature data for the left and right feet of the same user (subject or patient), similar to that for block 801 detailed above. The training phase continues at block 903, which is identical to blocks 403 and 803, as described above for FIG. 4 and FIG. 8.

In the operational phase, at block 911, dynamic temperature trends for both feet, left and right, are obtained, for example, from data acquired from system users, typically other diabetic patients at various stages of the disease. Temperature data is, for example, acquired from the overall system's sensors over time, during ambulation, at rest, or during other activities.

The process moves to block 912. The process of block 912 is an optional process, and need not be performed when preprocessing is not needed, for example, when raw data is suitable for use in the models. At block 912, when preprocessing is needed, the preprocessing steps to be performed include, for example, one or more of normalization, left and right foot spatial alignment, and, identification/removal/reconstruction of artifacts (artefactual data).

Next, using the determination/training of the classification/regression model of block 903, and from block 912 (if preprocessing steps are performed), or block 911 (if preprocessing steps were not necessary, and are not performed), the process moves to block 913. At block 913, the temperature data (with or without preprocessing) is input into the classification/regression model(s), to obtain vascular status (VS), thermoregulatory status (TS), and ulceration status (US), for example, by artificial intelligence (AI) methods, such as deep learning.

From block 913, the process moves to block 920, where it ends. The resultant data is now ready for analysis by the process of FIG. 12.

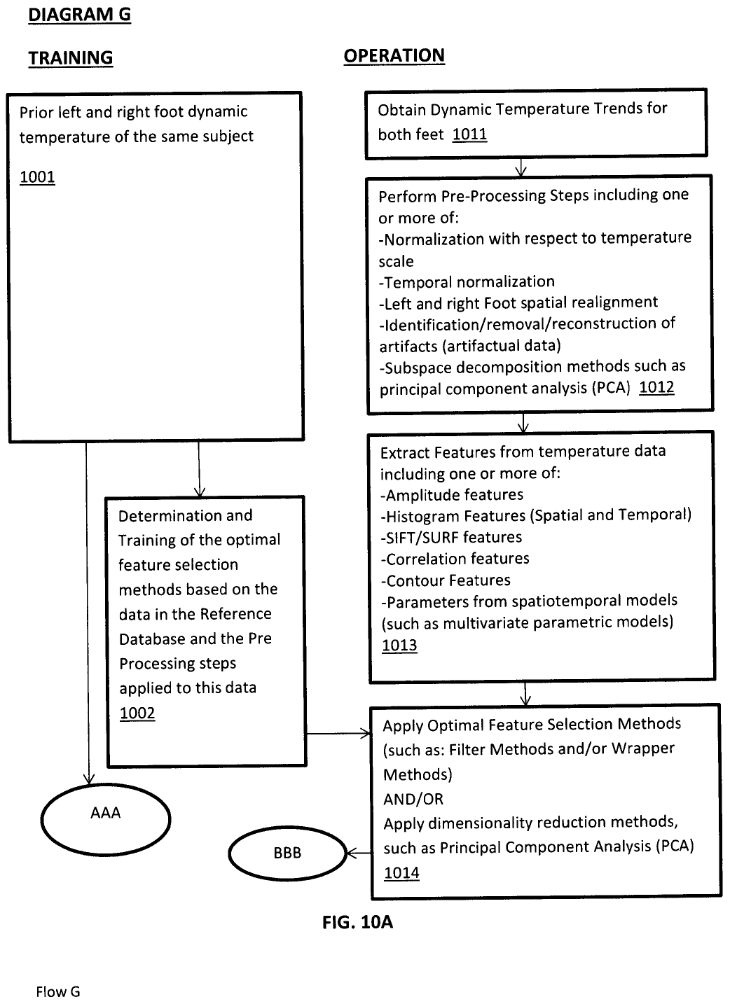

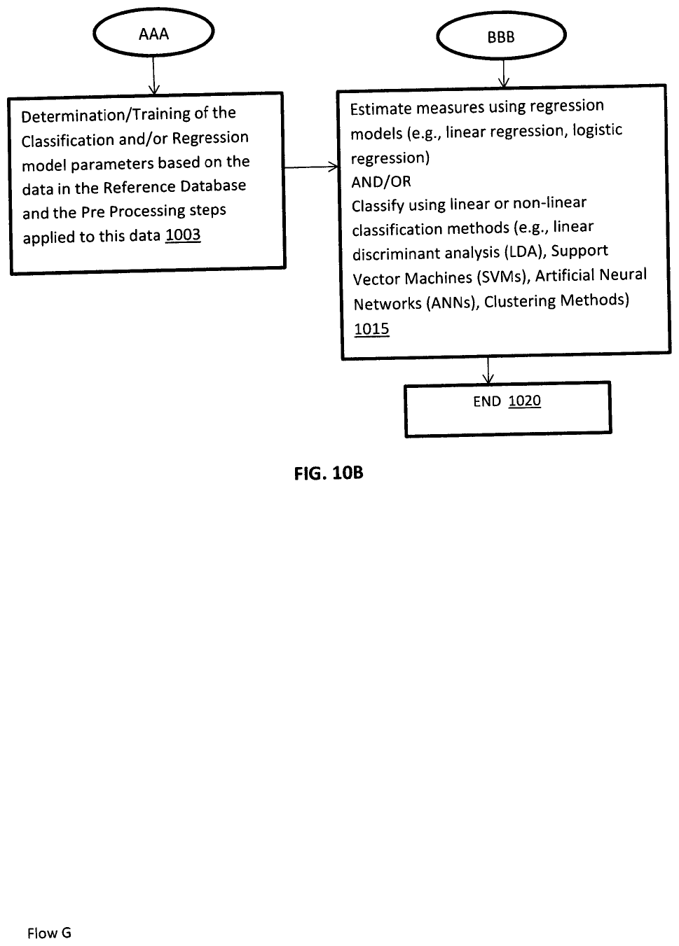

Attention is now directed to FIGS. 10A and 10B, collectively referred to as FIG. 10, and DIAGRAM G, a process for determining vascular progression levels, thermoregulatory progression levels, and ulceration progression levels, using dynamic data with respect to that of a population. The diagram includes a Training Phase at blocks 1001, 1002 and 1003 and an Operation Phase at blocks 1011-1015, terminating at an END, at block 1020.

The training phase, at block 1001, includes obtaining dynamic temperature data for the prior left and right foot dynamic temperature of the same user (subject or patient). The training phase continues at blocks 1002 and 1003, which are identical to blocks 402/602/802 and 403/603/803, respectively, as described for FIGS. 4, 6, and 8.

In the operational phase, on the right side of the page, the process includes blocks 1011, 1012, 1013, 1014 and 1015. These processes are similar to corresponding blocks 811, 812, 813, 814 and 815 of FIG. 8, respectively, and are in accordance with these blocks as described for FIG. 8 above.

From block 1015, the process moves to block 1020, where it ends. The resultant data is now ready for analysis by the process of FIG. 12.

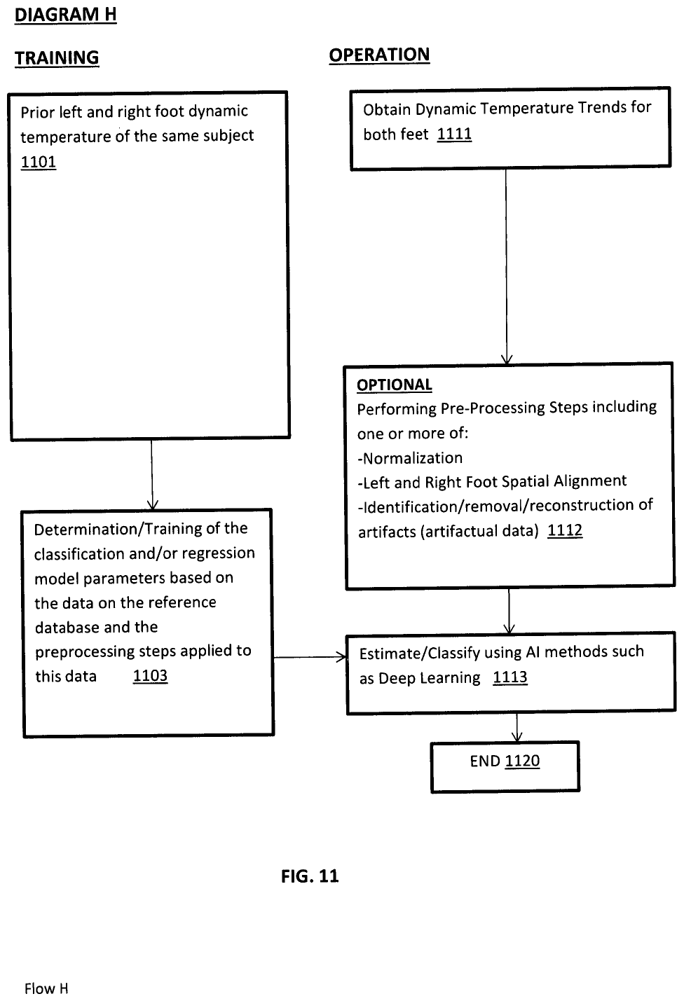

Attention is now directed to FIG. 11, and DIAGRAM G, a process for determining vascular progression levels, thermoregulatory progression levels, and ulceration progression levels, using dynamic data with respect to that of a population. The diagram includes a Training Phase at blocks 1101 and 1103, and an Operation Phase at blocks 1111-1113, terminating at an END, at block 1120.

The training phase, at block 1101, at the left side of the page, includes obtaining dynamic temperature data for the prior left and right foot dynamic temperature of the same user (subject or patient). The establishing of the databases and the actual databases are similar to those of block 1001 of the processes of FIG. 10, and is in accordance with that described above for FIG. 10.

The training phase moves from block 1101 to block 1103. Block 1103 is identical to blocks 403/603/803/903/1003, as described for FIGS. 4, 6, 8, 9 and 10, above.

In the operational phase, blocks 1111, 1112 and 1113 are similar to blocks 911, 912 and 913, respectively, and are in accordance with the descriptions of blocks 911, 912 and 913, from FIG. 9 above.

From block 1113, the process moves to block 1120, where it ends. The resultant data is now ready for analysis by the process of FIG. 12.

FIG. 12 is a flow diagram of a general process from processes (and corresponding Flow Diagrams) in DIAGRAM A through DIAGRAM H performed by the system 120′, for example, of the home server 120 and/or any peripheral components (as well as the mobile computer 110 and application server 126). The data produced by the processes of the DIAGRAM A through DIAGRAM H, and corresponding FIGS. 4 through 11, is received by the system 120′ (as well as the mobile computer 110 and application server 126), and processed in two steps.

In STEP 1, the received data is divided into Condition Status at block 1202, from data obtained from the user (as well as previous readings which are typically non-instantaneous), and Progression Level of the condition of the user, at block 1204, by iteratively adding this data to the user's prior data for the requisite condition. In STEP 2, a Foot Health Profile is made, and depending on the results of the profile, alerts/notifications/recommendations are issued to a recipient, for example, the patient or the associated health care professional.

The Condition Status (block 1202), which relates to a user's Vascular Status (VS), Thermoregulatory Status (TS), and Ulceration Status (US) is the condition, resulting from the diabetic conditions of vascular conditions, thermoregulatory conditions, and ulceration, respectively, of the user in relation to the population data and is as follows from most severe, to least severe as:

Condition Status
Severely Compromised
Moderately Compromised
Marginally Compromised
Uncompromised

The Progression Level (block 1204), is the level of improvement/deterioration for one of the conditions of: which relates to a user's Vascular Status (VS), Thermoregulatory Status (TS), and Ulceration Status (US), resulting from the diabetic conditions of vascular conditions, thermoregulatory conditions, and ulceration, respectively and, with respect to the user's own prior data and is as follows from most severe, to least severe as:

Progression Level
Severe Deterioration
Moderate Deterioration
No Change
Moderate Improvement
Strong Improvement

In order to arrive at each Condition Status and Progression Level, the various subprocesses of each of the processes of Flow DIAGRAMs A through H can be, and typically are weighted.

The process moves to block 1206, where a foot health profile is assembled for the particular user. Based on the condition status detailed above, the foot health profile includes Vascular Status (VS), Thermoregulatory Status (TS) and Ulcer Status (US). The Progression Levels of the profile include the Vascular Progression Level (VPL), the Thermoregulatory Progression Level (TPL) and the Ulcer Progression Level (UPL). Based on the foot health profile, for example, and condition being compromised and or moderate deterioration of the Vascular Progression Level, the Thermoregulatory Progression Level and the Ulcer Progression Level, alerts/notifications/recommendations will be provided, for example, transmitted, to a recipient, such as the user, the user's clinician, or the like, at block 1208.

Embodiments of the disclosed subject matter are directed to a method for determining a diabetic medical condition. The method comprises: determining, from foot data in a population: 1) a range of values indicative of the presence of a diabetic medical condition, and, 2) a plurality of levels within the range of values, each level corresponding to the status of the diabetic medical condition; obtaining foot data at a location on the foot of a patient; and, analyzing the obtained foot data to determine at least one value for determining whether the patient is experiencing the diabetic medical condition, and if experiencing the diabetic medical condition, the level of the status of the diabetic medical condition.

Optionally, the method is such that the diabetic medical condition includes one or more of: vascular conditions, thermoregulatory conditions, and ulceration. Optionally, the method is such that the level of the status of the diabetic medical condition includes at least one of: uncompromised, marginally compromised, moderately compromised, and severely compromised. Optionally, the method is such that the obtaining foot data at a location on the foot includes a plurality of locations on the foot, the locations including a plantar aspect of the foot and/or a dorsal aspect of the foot. Optionally, the method is such that the foot data includes temperature. Optionally, the method is such that the foot data includes one or more of temperature, acceleration and/or pressure. Optionally, the method is such that the temperature is obtained from temperature sensors. Optionally, the method is such that the temperature sensors are arranged in arrays comprising one or more of temperature sensors. Optionally, the method is such that the temperature is obtained from temperature sensors, the acceleration is obtained from accelerometers, and the pressure is obtained from pressure sensors. Optionally, the method is such that the temperature sensors, accelerometers, and/or the pressure sensors are arranged in arrays comprising one or more of the temperature sensors, accelerometers, and/or the pressure sensors. Optionally, the method is such that the temperature sensors are located on a wearable form factor configured to conform to the shape of a foot. Optionally, the method is such that the wearable form factor includes: a sock, an insole, wrap, shoe, ankle support, or other foot covering. Optionally, the method is such that the temperature sensors, the accelerometers and the pressure sensors are located on a wearable form factor configured to conform to the shape of the foot. Optionally, the method is such that the wearable includes: a sock, an insole, wrap, shoe, ankle support, or other foot covering. Optionally, the method is such that the determining the range of values indicative of the diabetic medical condition and the values indicating the plurality of levels of the status of the diabetic medical condition is performed by training a model. Optionally, the method is such that the analyzing is performed on the trained model. Optionally, the method is such that the model includes at least one of a classification model or a regression model. Optionally, the method is such that the values of 1) the range of values; and 2) the at least one value include normalized values.

Embodiments of the disclosed subject matter are directed to a method for determining a diabetic medical condition. The method comprises: determining, from foot data in a population: 1) a range of values over a first predetermined time period indicative of the presence of a diabetic medical condition, and, 2) a plurality of levels within the range of values over the first predetermined time period, each level corresponding to the progression of the diabetic medical condition; obtaining foot data at a location on the foot of a patient at a plurality of times within a second predetermined time period; and, analyzing at least two instances of the obtained foot data, to determine at least one value for each of the two instances, for determining whether the patient is experiencing the diabetic medical condition, and if experiencing the diabetic medical condition, the progression of the diabetic medical condition.

Optionally, the method is such that diabetic medical condition includes one or more of: vascular conditions, thermoregulatory conditions, and ulceration. Optionally, the method is such that the progression of the diabetic medical condition is determined as one of: strong improvement, moderate improvement, no change, moderate deterioration, and, severe deterioration. Optionally, the method is such that the measuring foot data at a location on the foot includes a plurality of locations on the foot, the locations including a plantar aspect of the foot and/or a dorsal aspect of the foot. Optionally, the method is such that the foot data includes temperature. Optionally, the method is such that the foot data includes one or more of temperature, acceleration and/or pressure. Optionally, the method is such that the temperature is obtained from temperature sensors. Optionally, the method is such that the temperature sensors are arranged in arrays comprising one or more of the temperature sensors. Optionally, the method is such that the temperature is obtained from temperature sensors, the acceleration is obtained from accelerometers, and the pressure is obtained from pressure sensors. Optionally, the method is such that the temperature sensors, accelerometers, and/or the pressure sensors are arranged in arrays comprising one or more of the temperature sensors, accelerometers, and/or the pressure sensors. Optionally, the method is such that the temperature sensors are located on a wearable form factor configured to conform to the shape of a foot. Optionally, the method is such that the wearable form factor includes: a sock, an insole, wrap, shoe, ankle support, or other foot covering. Optionally, the method is such that the temperature sensors, the accelerometers and the pressure sensors are located on a wearable form factor configured to conform to the shape of the foot. Optionally, the method is such that the wearable includes: a sock, an insole, wrap, shoe, ankle support, or other foot covering. Optionally, the method is such that the determining the range of values indicative of the diabetic medical condition and the values indicating the plurality of levels of the progression of the diabetic medical condition is performed by training a model. Optionally, the method is such that the analyzing is performed on the trained model. Optionally, the method is such that the model includes at least one of a classification model or a regression model. Optionally, the method is such that the values of 1) the range of values; and 2) the at least one value include normalized values.

Embodiments of the disclosed subject matter are directed to a system for determining a diabetic medical condition. The system comprises: a device including: a body, at least one temperature sensor configured on the body to be proximate to a foot, the at least one temperature sensor for measuring temperatures of the foot, and, a transmitter for transmitting the foot temperatures; and, a computer system. The computer system comprises: a receiver for receiving the transmitted foot temperatures; a storage medium for storing computer components; and, at least one processor for executing the computer components. The computer components comprise: a first module or converting the received foot temperatures into values; and, a second module for analyzing the values against: 1) a range of values indicative of the presence of a diabetic medical condition, to determine the presence of a diabetic medical condition; and, 2) a plurality of levels within the range of values, each level corresponding to the status of the diabetic medical condition, to determine the status of the diabetic medical condition.

Optionally, the system is such that the diabetic medical condition includes one or more of: vascular conditions, thermoregulatory conditions, and ulceration. Optionally, the system is such that the level of the status of the diabetic medical condition includes at least one of: uncompromised, marginally compromised, moderately compromised, and severely compromised. Optionally, the system is such that the body includes a form factor. Optionally, the system is such that the form factor includes: a sock, an insole, wrap, shoe, ankle support, or other foot covering. Optionally, the system is such that the at least one temperature sensor includes a plurality of temperature sensors arranged in one or more arrays, each of the one or more arrays including at least one temperature sensor and, the receiver for receiving the foot temperatures from each array of the plurality of arrays. Optionally, the system is such that the each of the arrays are positioned on the body to be in communication with a plantar portion of the foot and/or a dorsal portion of the foot. Optionally, the system is such that the body includes a form factor. Optionally, the system is such that the form factor includes: a sock, an insole, wrap, shoe, ankle support, or other foot covering. Optionally, the system is such that the first module for converting the received foot temperatures into values includes converting the received foot temperatures into normalized values. Optionally, the system is such that the body additionally comprises at least one of an additional sensor, including at least one pressure sensor and/or at least one accelerometer, additional sensor, the transmitter for transmitting data from the at least one additional sensor to the receiver, and the first module additionally configured to convert the received foot temperatures and the data from the at least one additional sensor into the normalized values.

Embodiments of the disclosed subject matter are directed to a system for determining a diabetic medical condition. The system comprises: a device including: a body, at least one temperature sensor configured on the body to be proximate to a foot, the at least one temperature sensor for measuring temperatures of the foot, and, a transmitter for transmitting the foot temperatures; and, a computer system. The computer system comprises: a receiver for receiving the transmitted foot temperatures; a storage medium for storing computer components; and, at least one processor for executing the computer components. The computer components comprise: a first module for converting the received foot temperatures into values; and, a second module for analyzing: 1) the values against a range of values indicative of the presence of a diabetic medical condition, to determine the presence of a diabetic medical condition; and, 2) at least a plurality of the values from within a first predetermined time period against a plurality of ranges of values over a second predetermined time period, to determine progression of the diabetic medical condition.

Optionally, the system is such that the diabetic medical condition includes one or more of: vascular conditions, thermoregulatory conditions, and ulceration. Optionally, the system is such that the progression of the diabetic medical condition is determined as one of: strong improvement, moderate improvement, no change, moderate deterioration, and, severe deterioration. Optionally, the system is such that the body includes a form factor. Optionally, the system is such that the form factor includes: a sock, an insole, wrap, shoe, ankle support, or other foot covering. Optionally, the system is such that the at least one temperature sensor includes a plurality of temperature sensors arranged in one or more arrays, each of the one or more arrays including at least one temperature sensor and, the receiver for receiving the foot temperatures from each array of the plurality of arrays. Optionally, the system is such that each of the arrays are positioned on the body to be in communication with a plantar portion of the foot and/or a dorsal portion of the foot. Optionally, the system is such that the body includes a form factor. Optionally, the system is such that the form factor includes: a sock, an insole, wrap, shoe, ankle support, or other foot covering. Optionally, the system is such that the first module for converting the received foot temperatures into values includes converting the received foot temperatures into normalized values. Optionally, the system is such that the body additionally comprises at least one of an additional sensor, including at least one pressure sensor and/or at least one accelerometer, additional sensor, the transmitter for transmitting data from the at least one additional sensor to the receiver, and the first module additionally configured to convert the received foot temperatures and the data from the at least one additional sensor into the normalized values.

Implementation of the method and/or system of embodiments of the disclosed subject matter can involve performing or completing selected tasks manually, automatically, or a combination thereof. Moreover, according to actual instrumentation and equipment of embodiments of the method and/or system of the disclosed subject matter, several selected tasks could be implemented by hardware, by software or by firmware or by a combination thereof using an operating system.

For example, hardware for performing selected tasks according to embodiments of the disclosed subject matter could be implemented as a chip or a circuit. As software, selected tasks according to embodiments of the disclosed subject matter could be implemented as a plurality of software instructions being executed by a computer using any suitable operating system. In an exemplary embodiment of the disclosure, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, non-transitory storage media such as a magnetic hard-disk and/or removable media, for storing instructions and/or data. Optionally, a network connection is provided as well. A display and/or a user input device such as a keyboard or mouse are optionally provided as well.

For example, any combination of one or more non-transitory computer readable (storage) medium(s) may be utilized in accordance with the above-listed embodiments of the present disclosure. The non-transitory computer readable (storage) medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

As will be understood with reference to the paragraphs and the referenced drawings, provided above, various embodiments of computer-implemented methods are provided herein, some of which can be performed by various embodiments of apparatuses and systems described herein and some of which can be performed according to instructions stored in non-transitory computer-readable storage media described herein. Still, some embodiments of computer-implemented methods provided herein can be performed by other apparatuses or systems and can be performed according to instructions stored in computer-readable storage media other than that described herein, as will become apparent to those having skill in the art with reference to the embodiments described herein. Any reference to systems and computer-readable storage media with respect to the following computer-implemented methods is provided for explanatory purposes, and is not intended to limit any of such systems and any of such non-transitory computer-readable storage media with regard to embodiments of computer-implemented methods described above. Likewise, any reference to the following computer-implemented methods with respect to systems and computer-readable storage media is provided for explanatory purposes, and is not intended to limit any of such computer-implemented methods disclosed herein.

The flowcharts and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

The above-described processes, including portions thereof can be performed by software, hardware and combinations thereof. These processes and portions thereof can be performed by computers, computer-type devices, workstations, processors, micro-processors, other electronic searching tools and memory and other non-transitory storage-type devices associated therewith. The processes and portions thereof can also be embodied in programmable non-transitory storage media, for example, compact discs (CDs) or other discs including magnetic, optical, etc., readable by a machine or the like, or other computer usable storage media, including magnetic, optical, or semiconductor storage, or other source of electronic signals.

The processes (methods) and systems, including components thereof, herein have been described with exemplary reference to specific hardware and software. The processes (methods) have been described as exemplary, whereby specific steps and their order can be omitted and/or changed by persons of ordinary skill in the art to reduce these embodiments to practice without undue experimentation. The processes (methods) and systems have been described in a manner sufficient to enable persons of ordinary skill in the art to readily adapt other hardware and software as may be needed to reduce any of the embodiments to practice without undue experimentation and using conventional techniques.

Although the disclosed subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for determining a diabetic medical condition comprising:
determining, from foot data in a population: 1) a range of values indicative of the presence of a diabetic medical condition, and, 2) a plurality of levels within the range of values, each said level corresponding to the status of the diabetic medical condition;
obtaining foot data at a location on the foot of a patient via a wearable device; and,
analyzing the obtained foot data to determine at least one value for determining whether the patient is experiencing the diabetic medical condition, and if experiencing the diabetic medical condition, the level of the status of the diabetic medical condition.

2. (canceled)

3. The method of claim 1, wherein the level of the status of the diabetic medical condition includes at least one of: uncompromised, marginally compromised, moderately compromised, and severely compromised.

4.-14. (canceled)

15. The method of claim 1, wherein the determining the range of values indicative of the diabetic medical condition and the values indicating the plurality of levels of the status of the diabetic medical condition is performed by training a model or using a pre-trained model.

16.-18. (canceled)

19. A method for determining a diabetic medical condition comprising:
determining, from foot data in a population: 1) a range of values over a first predetermined time period indicative of the presence of a diabetic medical condition, and, 2) a plurality of levels within the range of values over the first predetermined time period, each said level corresponding to the progression of the diabetic medical condition;
obtaining foot data at a location on the foot of a patient via a wearable device at a plurality of times within a second predetermined time period; and,
analyzing at least two instances of the obtained foot data, to determine at least one value for each of the two instances, for determining whether the patient is experiencing the diabetic medical condition, and if experiencing the diabetic medical condition, the progression of the diabetic medical condition.

20. (canceled)

21. The method of claim 1, wherein the diabetic medical condition includes one or more of: vascular conditions, thermoregulatory conditions, and ulceration; and, the progression of the diabetic medical condition includes one of: strong improvement, moderate improvement, no change, moderate deterioration, and, severe deterioration.

22. The method of claim 1, wherein the measuring foot data at a location on the foot includes a plurality of locations on the foot, the locations including a plantar aspect of the foot and/or a dorsal aspect of the foot.

23. (canceled)

24. The method of claim 22, wherein the foot data includes one or more of temperature, acceleration and/or pressure.

25. The method of claim 24, wherein the temperature is obtained from temperature sensors arranged in arrays comprising one or more of said temperature sensors, the acceleration is obtained from accelerometers, and the pressure is obtained from pressure sensors on the wearable.

26.-29. (canceled)

30. The method of claim 1, wherein the wearable includes: a sock, an insole, wrap, shoe, ankle support, or other foot covering.

31.-32. (canceled)

33. The method of claim 19, wherein the determining the range of values indicative of the diabetic medical condition and the values indicating the plurality of levels of the progression of the diabetic medical condition is performed by training a model or using a pre-trained model.

34.-36. (canceled)

37. A system for determining a diabetic medical condition comprising:
a wearable device for the foot including: a body, at least one temperature sensor configured on the body to be proximate to a foot, the at least one temperature sensor for measuring temperatures of the foot, and, a transmitter for transmitting the foot temperatures; and,
a computer system comprising: a receiver for receiving the transmitted foot temperatures; a storage medium for storing computer components; and, at least one processor for executing the computer components comprising: a first module or converting the received foot temperatures into values; and, a second module for analyzing:
1) the values against a range of values indicative of the presence of a diabetic medical condition, to determine the presence of a diabetic medical condition; and,
2) at least one of:
at least a plurality of the values from within a first predetermined time period against a plurality of ranges of values over a second predetermined time period, to determine progression of the diabetic medical condition; or,
a plurality of levels within the range of values, each said level corresponding to the status of the diabetic medical condition, to determine the status of the diabetic medical condition.

38. The system of claim 37, wherein the diabetic medical condition includes one or more of: vascular conditions, thermoregulatory conditions, and ulceration; and,
if there is a diabetic medical condition, the progression of the diabetic medical condition is determined as one of: strong improvement, moderate improvement, no change, moderate deterioration, and, severe deterioration; or
the level of the status of the diabetic medical condition includes at least one of: uncompromised, marginally compromised, moderately compromised, and severely compromised.

39.-40. (canceled)

41. The system of claim 37, wherein the wearable includes: a sock, an insole, wrap, shoe, ankle support, or other foot covering.

42. The system of claim 37, wherein the system includes a plurality of temperature sensors arranged in one or more arrays on the body, each of the one or more arrays including at least one temperature sensor and, the receiver for receiving the foot temperatures from each array of the plurality of arrays.

43.-46. (canceled)

47. The system of claim 37, wherein the body additionally comprises;
at least one of an additional sensor, including one or more of a pressure sensor and/or at least one accelerometer, and,
the transmitter for transmitting data from the at least one additional sensor to the receiver, and the first module additionally configured to convert the received foot temperatures and the data from the at least one additional sensor into the said normalized values.

48.-58. (canceled)
